(19) United States
(12) Patent Application Publication
(10) Pub. No.: US 2003/0216623 A1
(43) Pub. Date: Nov. 20, 2003

(54) Method and system for supporting therapy planning

(76) Inventors: Klaus Abraham-Fuchs, Erlangen (DE); Uwe Eisermann, Erlangen (DE); Niels Richter, Thurnau (DE); Robert Setz, Rednitzhembach (DE)

Correspondence Address: HARNESS, DICKEY & PIERCE, P.L.C., P.O.BOX 8910, RESTON, VA, 20195, US

(21) Appl. No.: 10/431,538

(22) Filed: May 8, 2003

(30) Foreign Application Priority Data
May 8, 2002 (EP) 02010468.3

(51) Int. Cl. A61B 5/00

(52) U.S. Cl. 600/300

(57) ABSTRACT

A method and a system is for supporting therapy planning, particularly in rehabilitation. The method involves a capability profile, including a plurality of capability deficits, for a patient at the start of a therapy and/or information about therapy modules prescribed for the patients in the course of therapy being provided from a database. Each of the capability deficits and/or therapy modules is automatically assigned one or more organization units by using a second database which contains, for a plurality of capability deficits and/or therapy modules, a fixed link to organization units providing therapy. At least some of the capability deficits and/or therapy modules are displayed with their association or on the basis of their association on a computer workstation. The method and the associated system simplify therapy planning and therapy progress control for the treating physician or therapist.

The present application hereby claims priority under 35 U.S.C. §119 on European patent application number EP 02010468.3 filed May 8, 2002, the entire contents of which are hereby incorporated herein by reference.

### FIELD OF THE INVENTION

The present invention generally relates to a method and a system for supporting therapy planning, particularly in rehabilitation.

### BACKGROUND OF THE INVENTION

Serious illnesses such as stroke, heart attack or Alzheimer's disease or serious operations such as the insertion of joint implants or the performance of an amputation cause most patients to have different deficits in physical and mental performance. These deficits are generally the result of the weakening or complete failure of a region of the brain or of a muscle. Combinations of these also frequently arise. Thus, by way of example, a region of the brain which is responsible for controlling a muscle or a number of muscles in functional chains can be damaged. As a result, the affected muscles degenerate, which means that they are no longer able to be used properly.

Such mental or physical restrictions are referred to in the medical vernacular as capability deficits, which can be split into various areas of capability. Thus, one known classification makes the following exemplary distinctions:

motor capabilities such as strength, stamina, mobility, balance, reaction, orientation, differentiation, accommodation, speech motor functions;

intellectual/cognitive capabilities such as attention, memory, planning, comprehension of speech, communication, vision;

organic/physical capabilities such as reduction of organ performance;

social capabilities such as ability to communicate and participate;

emotional capabilities such as the capability to develop self-esteem.

Some capabilities also require interplay between motor functions and cognitive functions. Thus, by way of example, the activity of climbing stairs requires strength and balance as motor capabilities and attention and spatial awareness as cognitive capabilities.

Very often, a patient does not have a single deficit in one capability category but rather has a combination of a plurality of deficits in a more or less serious form. The aim of a therapeutic measure, which is normally performed as part of a rehabilitation process, is to restore the capabilities or to reduce the existing deficits as far as possible. At the start of the rehabilitative measure, this generally involves all the patient's capability deficits being recorded using known methods of measurement, observation and questioning, and their extent being documented. This recording process is also referred to as staging the patient. Depending on the methods of measurement used, the result of this staging process is quantitative, for example a percentage of visual capability or an indication of the degree of mobility in the upper arm, or qualitative, for example a classification of the capability restriction as severe, intermediate or slight. One example of an established method of measurement for staging numerous neurological, cognitive and physical capabilities is the “Wiener test series” from the company Schuhfried.

The result of this initial examination is ideally a cross-discipline capability report which can be presented in the form of a capability profile. In this context, a capability profile is defined as a list of all relevant capabilities and an association between the degree of the restriction in these capabilities for this patient and the time at which the information was collected.

In addition to the term capabilities, the term skill is also used in the medical vernacular. In the context of a medical rehabilitation measure, a skill is understood to mean a complex action but one which is self-contained and can be delimited with respect to other actions. A skill requires interplay between a plurality of capabilities. In particular, the term skill in the context of rehabilitation refers to activities of daily living (ADL) which are a primary prerequisite for independent, autonomous living. Examples of such skills are eating, dressing, washing, showering, climbing stairs, etc. The performance of such skills is also recorded in standardized questionnaires and is quantified as an ADL index. Although rehabilitation directly involves the training of capabilities, the actual aim is to reacquire skills. In this respect, the terms capability and skill can normally be interchanged within the context of the description below.

Normally, patients simultaneously have a plurality of capability deficits which come under the area of responsibility and competence of various organization units providing therapy, such as different specialist departments at a rehab clinic or at a hospital. In this case, coordinating the therapy planning and controlling the progress of therapy between the individual organization units is a time-consuming and work-intensive task. This task does not need to be performed just once at the start of therapy but rather requires continuous coordination between the individual organization units throughout the progress of therapy. To date, this coordination has been effected by means of numerous appointments, oral consultations and the exchange of written documents between the relevant organization units at the hospital or at the rehab clinic, between the treating physicians and therapists and possibly between different service providers in an integrated health service.

### SUMMARY OF THE INVENTION

An object of an embodiment of the present invention is to specify a method and a system for supporting therapy planning, particularly in rehabilitation, which significantly simplify therapy planning in terms of the time taken and the workload for the individual physician or therapist.

An object may be achieved by a method and/or a system. Advantageous refinements of the method and of the system can be found in the description below and in the exemplary embodiments.

In one embodiment, the present method involves a capability profile, including a plurality of capability deficits, for a patient at the start of a therapy and/or information about therapy modules prescribed for the patient in the course of therapy being provided from a first database. Each of the capability deficits and/or therapy modules is automatically assigned one or more organization units by using a second database which contains, for a large number of capability deficits and/or therapy modules, a prescribed link to organization units providing therapy. Next, at least some of the capability deficits and/or therapy modules are displayed with their association or on the basis of their association on a computer workstation. In this context, display on the basis of their association means that the information displayed is selected on the basis of the association with organization units, but the organization unit itself is not displayed at the same time.

The associated system for supporting therapy planning comprises a computer workstation with access to a first database, which includes a capability profile, including a plurality of capability deficits, for a patient and/or information about therapy modules prescribed for the patient, and to a second database, which contains, for a large number of capability deficits and/or therapy modules, a fixed link to organization units providing therapy. The system also includes a module which retrieves the capability deficits and/or therapy modules from the first database, automatically assigns each of the capability deficits and/or therapy modules one or more organization units by using the second database, and displays at least some of the capability deficits and/or therapy modules with their association or on the basis of their association on the computer workstation.

The present method and the associated system provide the user, particularly the physician or therapist, with computer-based IT tools for visualizing patient status and therapy process across departments. This enables the doctor or therapist to use the computer workstation to plan a therapy with efficient use of time and optimum coordination without the need for numerous appointments and oral consultations with other organization units which are involved. A clear graphical presentation of all or at least some of the patient's capability deficits and/or therapy modules in conjunction with the associated organization units providing therapy in the different categories simplifies therapy planning considerably. The presentation of the association between the patient's capability deficits and/or therapy modules and organization units can additionally be broken down according to organization categories, such as individual institutions in the health service (e.g. rehab clinic, hospital, medical practice, physiotherapy practice), medical specialist disciplines or specialist departments at a hospital.

In one development of the present method, a current level of the patient's capability deficits and/or a current performance level of the patient in the individual therapy modules is/are additionally retrieved from the first or from a further database and is/are displayed on the computer workstation. Thus, the physician or therapist also has an overview of the current level of and the alteration in the deficits in areas which are not part of his specialist field at all times in the course of the treatment period. In this context, the current performance level of the patient in the individual therapy modules is preferably represented by at least one dimension which represents the progress of therapy. Such a dimension can be obtained, by way of example, by measuring the performance of the patient upon carrying out a computer-assisted exercise using computer evaluation of the patient's inputs, by recording sensor signals on a training device or by evaluating entries in patients' medical diaries. One example of the recording of dimensions can be found in U.S. Pat. No. 6,261,239, for example.

In another refinement of the present method, the capability deficits and/or therapy modules are automatically assigned administration information by using a third database which contains, for a large number of capability deficits and/or therapy modules, a fixed link to associated administration information, and said assigned administration information is displayed on the computer workstation in conjunction with the capability deficits and/or therapy modules. This refinement serves to simplify administration work, such as the billing of health insurance companies. In this context, each individual capability can be assigned to the administration criteria, such as to the billing codes ICD or DRG which are customary in Germany.

In addition, the present method allows the association of the progress of therapy, responsibilities and administration criteria to be retrieved from the databases just for a single therapy module and to be made available as a graphical presentation in the work process on the computer workstation.

In one development of the present method, a compliance, recorded in the course of rehabilitation, for the patient in the individual therapy modules is retrieved from the first or from a further database and is displayed on the computer workstation in conjunction with the associated therapy modules. Preferably, improved clarity of the presentation is achieved by displaying only that subset of information which is relevant in the respective work step. Thus, by way of example, only the presentation and association of the specialist departments involved are displayed if this work step involves time planning for the resources within the rehab clinic. The selection regarding which section of information for the association of responsibilities is displayed can be made by the user himself manually in one embodiment of the present invention. In a preferred embodiment of the present method and of the associated system, this selection is made automatically on the basis of context, however, and is adjusted when the work step changes. In the case of the present method, context-dependent presentation or selection is to be understood to mean selection on the basis of the respective work step which the user is currently performing on the computer workstation. When, by way of example, the user changes from working on one substep in the therapy planning process to another, the information about responsibilities which is provided on the screen automatically changes.

In another refinement of the present method and of the associated system, the information relevant to the respective work step is displayed on the screen in an arrangement and with graphical highlighting such that it is particularly easy to identify. Using the example of the work step for planning resources within the clinic, this would be consideration of the responsibility of further specialist departments, which are then given particular highlighting in the presentation.

The patient's capability deficits and/or therapy modules and possibly the current level of the capability deficits and/or the current performance level of the patient in the therapy modules and also possibly other information are preferably displayed on the basis of the organization unit with which the computer workstation is associated or which is indicated by a user. This refinement of the present method takes account of the fact that different subsidiary aspects from the entire collection of the available information are significant to physicians and therapists in different disciplines. In this embodiment, only the information which is important to the respective user is shown therefore. This can be done on the basis of the user's input, in which he indicates his discipline or his association with one organization unit or category, or by virtue of appropriate preconfiguration of the computer workstation on the basis of the organization unit in which it is being used.

In one preferred embodiment of the present method, an alteration in the level of the patient's capability deficits and/or in the performance level of the patient in the therapy modules over time is displayed on the computer workstation for a prescribable period of time within the therapy period. Thus, the current level of the therapy and the progress over relevant periods of time can be clearly visualized on the computer workstation, embedded in the work process of therapy planning and therapy progress control. Besides visualization of the progress over time and of the alteration in the level of the patient's capability deficits and/or performance over time, it is optionally also possible to form differences between the respective values at two times. By way of example, the difference between the current level and the start of therapy or between the current level and the level upon the last change to the prescribed training can be calculated and displayed on the screen. In this context, any time periods which can be prescribed by the user can be shown as a difference. In the same way, trend displays or variations over time can be visualized for a plurality of successive measurement times over a selected period. This provides the user with a large number of presentation options supporting him in therapy planning and therapy progress control. It goes without saying that the individual refinements of the present method and of the associated system can also be combined with one another as required.

The present method uses databases and graphical user interfaces on a computer workstation in order to provide a treating physician or therapist, who is usually responsible just for some of the capability deficits, with clear information about the level of treatment for other capability deficits and about other competent organization units involved in this patient's therapy process quickly and without additional complexity in the work process of therapy planning and therapy progress control for treating capability deficits. Databases which are used in the method and in the system at least in some of the embodiments and which are connected to the computer workstation comprise the patient's capability profile at the start of treatment, the patient's capability profiles at further times in the course of treatment, the therapy modules prescribed for the patient, the progress of therapy, quantified by a dimension, currently achieved in these therapy modules and also possibly the quantified compliance of the patient in the therapy modules, and the association between each capability and each therapy module and the responsible organization unit in various organization categories.

### BRIEF DESCRIPTION O THE DRAWINGS

The present method and the associated system are explained again briefly below with reference to exemplary embodiments in conjunction with the drawings, in which:

FIG. 1 shows an example of a capability profile for a patient;

FIG. 2 shows an example of the association between the patient's capability deficits and organization units providing therapy, broken down according to organization categories;

FIG. 3 shows an example of the association between the patient's capability deficits and administration categories;

FIG. 4 shows an example of the association between therapy modules prescribed for the patient and organization units, broken down according to organization categories;

FIG. 5 shows an overview of a system for supporting therapy planning using the present method;

FIG. 6 shows an example of the presentation of the performance level achieved in individual therapy modules as compared with the performance level at the start of treatment;

FIG. 7 shows an example of the presentation of the level achieved for the capability deficits as compared with the deficits at the start of treatment; and

FIG. 8 shows an example of the visualization of a capability deficit's variation over time since the start of treatment.

### DETAILED DESCRIPTION OF THE PREFERRED EMBODIMENTS

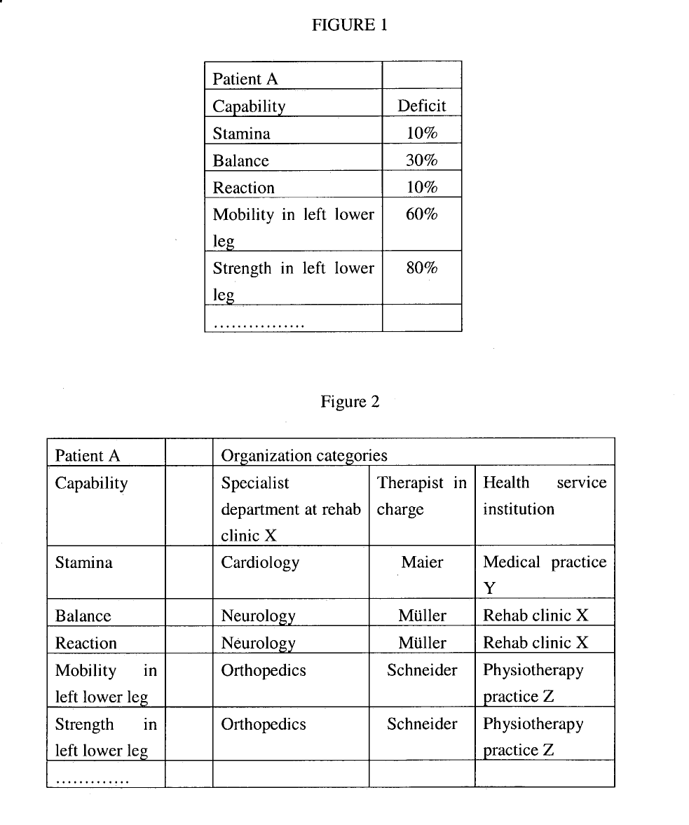

The starting point for the present method is a computer workstation 10 with access to a database 1 which contains the capability profile for a patient including quantification of the degree of each deficit after the initial examinations have been completed. An example of such a capability profile for a patient is shown in FIG. 1. The capability profile shown in this figure comprises the capabilities of stamina, balance, reaction, mobility in the left lower leg and strength in the left lower leg. The respective deficit is indicated as a percentage of the corresponding 100% capability in a healthy comparative person.

In addition, a second database 2 is implemented which contains, for a large number of capabilities, an association with organization units providing therapy, so that each capability appearing in the process can be assigned to a competent organization unit by accessing the second database 2. This database 2 can also contain an association with one or more organization categories. In this context, the organization categories are to be understood to be a generic term relating to organization units. The corresponding networking between the computer workstation 10 and the two databases 1, 2 is shown schematically in FIG. 5. In this case, the computer workstation 10 contains the modules 11, 12; of these, module 11 retrieves the capability deficits from the first database 1 and automatically assigns each of the capability deficits one or more organization units by using the second database 2, and module 12 graphically displays at least some of the capability deficits with their association on a monitor on the computer workstation 10.

FIG. 2 shows an example of the association between individual capability deficits and organization units. In this presentation, the organization units are broken down according to organization categories, particularly according to specialist departments at the rehab clinic X, the therapist responsible and the institution in the health service. This presentation immediately shows the treating physician or therapist which colleagues in other disciplines are additionally involved in the patient's rehabilitation measure.

The database 2 mapping the individual capabilities onto organization categories or organization units which are responsible for them is specific to the perspective of a respective institution which is under consideration. Hence, in the case of the computer workstation solution for improved workflow control proposed here, this database can be implemented individually for each organization.

Since the first database 1 containing the profile of the capability deficits is patient-specific, whereas the second database 2 containing the map between capabilities and the responsibility of various organization categories and units is institution-dependent, it is appropriate to separate these databases. Confidential patient data should be stored in an electronic patient record (EPR), while organization-specific data can be stored in a hospital information system (HIS). It goes without saying that the latter database can also be integrated in the computer workstation. Both databases can also be in the form of subsidiary databases of a larger database, however.

The present method involves the illustrated databases being used to provide a physician or therapist, who is typically responsible just for some of the capability deficits, with clear information about other competent organization units involved in this patient's therapy process on a computer workstation quickly and without additional complexity for him in the work process of therapy planning and therapy progress control. For this purpose, any arrangement and subset information from the lists shown in FIGS. 1 and 2 can be suitable.

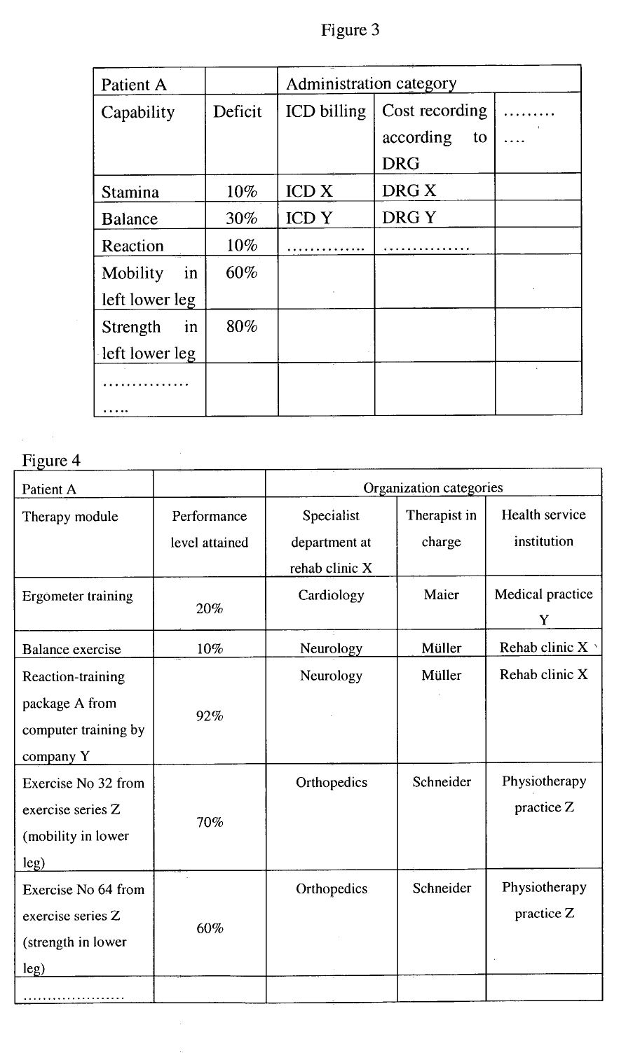

In addition, the computer workstation 10 can be connected to a further database 3, which contains an association between a large number of capabilities and administration categories. The modules 11, 12 in the computer workstation 10 then automatically assign the capabilities in the patient's capability profile to the administration categories and display these graphically as well, as can be seen in FIG. 3, for example. This figure shows the patient's capability deficits in association with the administration categories of billing according to ICD and cost recording according to DRG.

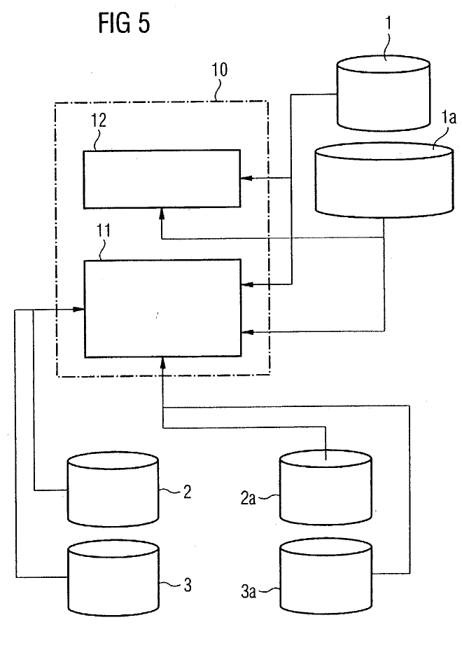

In addition to or instead of the database 1 containing the individual capability profile, the present method and the associated system can also involve a database 1a containing the therapy modules prescribed for the patient and the associated performance level and possibly the compliance in the individual therapy modules. In the same way as already explained in connection with the capability deficits, this refinement involves the module 11 in the computer workstation 10 assigning each of the therapy modules an organization unit by using a database 2a which contains a corresponding association between therapy modules and organization units in organization categories. An example of such an association is shown in FIG. 4, where the different therapy modules, for example ergonometer training or a reaction training package, have been assigned the corresponding organization units providing therapy, broken down according to organization categories. This presentation simultaneously shows the performance level attained in the individual therapy modules as a quantitative dimension, in this case as a percentage of the 100% performance of a healthy comparative person. In the case of this refinement too, an appropriate database 3a containing an association between a large number of therapy modules and administration categories can be used to assign the respective therapy modules prescribed for the patient to the appropriate administration categories and to display them as appropriate.

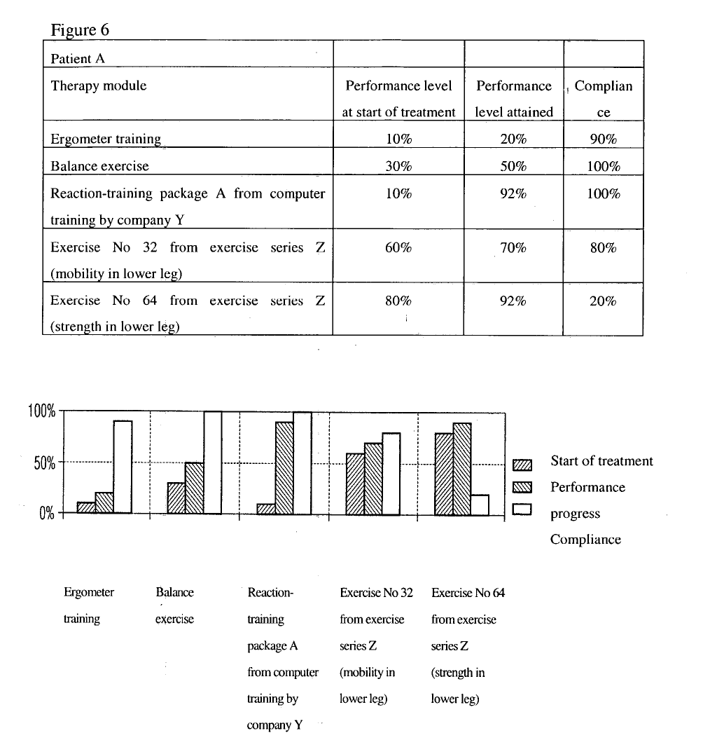

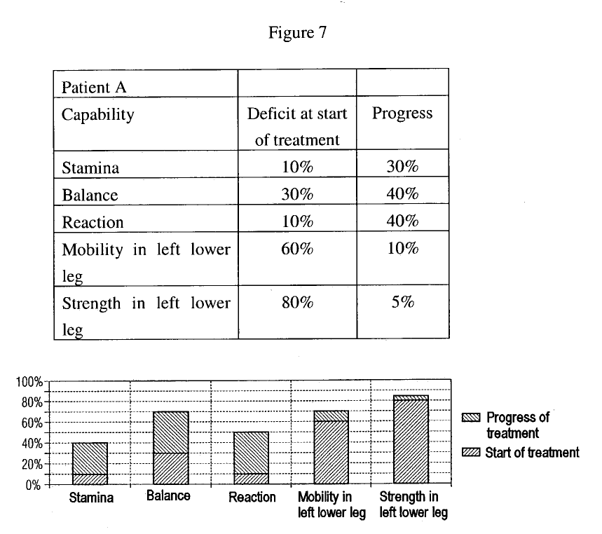

Besides the respective performance level attained, the database 1a can also contain a quantified dimension for the patient's compliance in the respective therapy module. This information can be used for a large number of different presentations. FIG. 6 shows an example of a presentation in which, for the different therapy modules prescribed for the patient, the performance level at the start of treatment is compared with the performance level currently attained and with the compliance. In the same way, the available databases for the capability profiles and therapy modules can be used with quantified therapy progress and compliance in order to provide clear visualization of the current level of the therapy and the progress over relevant periods of time on the computer workstation. The quantified values for capability deficit, performance level in an exercise or compliance can be presented as a bar graph or using a color scale, for example. Of particular advantage is the overview of the entire picture of the treatment process which the common database for all capability deficits and all therapy modules allows. In this regard, FIG. 7 shows a further example in which the capability deficits at the start of treatment are compared with the progress attained to date.

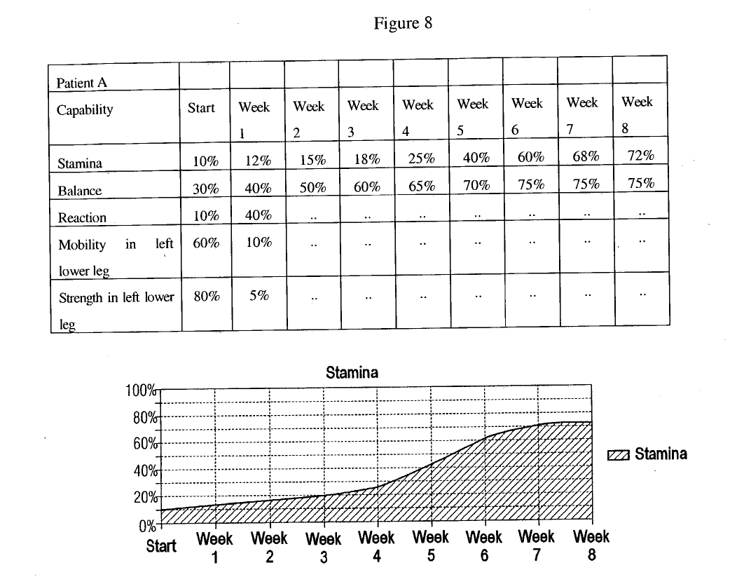

FIG. 8 shows an example of another presentation option in the present method and the associated system. In this exemplary embodiment, the variation in a capability deficit, in this case stamina, over time from the start of therapy up to the current recording time is visualized. It goes without saying that it is also possible to visualize other capability deficits, the performance level in individual therapy modules and the associated compliance in a comparable manner.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A method for supporting therapy planning, comprising:
providing, from a database, at least one of a capability profile, including a plurality of capability deficits for a patient at the start of a therapy, and information about therapy modules prescribed for the patient in the course of therapy;
automatically assigning each of the at least one of capability deficits and therapy modules, at least one organization unit, using a second database containing, for a plurality of at least one of capability deficits and therapy modules, a fixed link to organization units providing therapy; and
displaying at least one of the capability deficits and therapy modules, at least one of with their association and on the basis of their association, on a computer workstation.

2. The method as claimed in claim 1, wherein the association and display of the association between at least one of the patient's capability deficits and therapy modules and organization units are broken down according to superordinate organization categories.

3. The method as claimed in claim 1, wherein a current level of at least one of the patient's capability deficits and a current performance level of the patient in the therapy modules is retrieved from at least one of the first and from a further database and is displayed on the computer workstation.

4. The method as claimed in claim 3, wherein the current performance level of the patient in the therapy modules is represented by at least one dimension which represents the progress of therapy.

5. The method as claimed in claim 1, wherein at least one of the capability deficits and therapy modules are automatically assigned administration information by using a third database containing, for a plurality of at least one of capability deficits and therapy modules, a fixed link to associated administration information, and wherein the assigned administration information is displayed on the computer workstation.

6. The method as claimed in claim 1, wherein a recorded compliance for the patient in the therapy modules is retrieved from at least one of the first and from a further database and is displayed on the computer workstation in conjunction with the therapy modules.

7. The method as claimed in claim 1, wherein at least one of the patient's capability deficits and therapy modules are displayed on the basis of at least one of context and a user's selection.

8. The method as claimed in claim 1, wherein information relevant to the respective work step is graphically highlighted in the presentation.

9. The method as claimed in claim 1, wherein at least one of the patient's capability deficits and therapy modules are displayed on at least one of the basis of the organization unit with which the computer workstation is associated and which is specified by a user.

10. The method as claimed in claim 1, wherein, on the basis of the input by a user, just a single therapy module is displayed with at least one of the associated organization unit, with the progress of therapy, with the administration information and with the compliance.

11. The method as claimed in claim 1, wherein an alteration in at least one of the level of the patient's capability deficits and in the performance level of the patient in the therapy modules over time is displayed on the computer workstation for a prescribable period of time within the therapy period.

12. The method as claimed in claim 1, wherein, following two times being prescribed within the therapy period, a difference in at least one of the level of the patient's capability deficits, in the performance level of the patient in the therapy modules, and in the compliance is calculated between the two times and is displayed on the computer workstation in conjunction with at least one of the capability deficits and therapy modules.

13. A system for supporting therapy planning, comprising:
a computer workstation, including access to a first database containing at least one of a plurality of capability deficits for a patient and information about therapy modules prescribed for the patient, and including access to a second database containing, for a plurality of at least one of capability deficits and therapy modules, a fixed link to organization units providing therapy; and
a module, adapted to retrieve at least one of the capability deficits and therapy modules from the first database, adapted to automatically assign each of the at least one of capability deficits and therapy modules at least one organization unit in conjunction with the second database, and adapted to control display of at least one of the capability deficits and therapy modules, at least one of with their association and on the basis of their association, on the computer workstation.

14. The system as claimed in claim 13, wherein at least one of the first and second database is integrated in the computer workstation.

15. The system as claimed in claim 13, wherein at least one of the first and second database is connected to the computer workstation via a network.

16. The system as claimed in claim 13, wherein at least one of the first and a further database to which the computer workstation is connected contains at least one of a current level of the patient's capability deficits and a current performance level of a patient in the therapy modules, and wherein the module is designed such that it retrieves at least one of the current level of the patient's capability deficits and the current performance level of the patient in the therapy modules, at least one of automatically and on the basis of an input by a user, and is adapted to display it on the computer workstation.

17. The system as claimed in claim 13, wherein at least one of the second and a third database connected to the computer workstation, for a plurality of at least one of capability deficits and therapy modules, contains a fixed link to associated administration information, and wherein the module is adapted to retrieve the administration information at least one of automatically and on the basis of an input by a user and is adapted to display it on the computer workstation in conjunction with at least one of the capability deficits and therapy modules.

18. The system as claimed in claim 13, wherein the module is designed such that, on the basis of an input by a user, it is adapted to ascertain an alteration in at least one of the level of the patient's capability deficits and in the performance level of the patient in the therapy modules over time for a prescribable period of time by using the first database, and is adapted to display it on the computer workstation.

19. The method as claimed in claim 1, wherein the method is for supporting therapy planning in rehabilitation.

20. The method as claimed in claim 2, wherein a current level of at least one of the patient's capability deficits and a current performance level of the patient in the therapy modules is retrieved from at least one of the first and from a further database and is displayed on the computer workstation.

21. The method as claimed in claim 1, wherein at least one of the capability deficits and therapy modules are automatically assigned administration information by using a third database containing, for a plurality of at least one of capability deficits and therapy modules, a fixed link to associated administration information, and wherein the assigned administration information is displayed on the computer workstation in conjunction with at least one of the capability deficits and therapy modules.

22. The method as claimed in claim 1, wherein at least one of the patient's capability deficits and therapy modules, and at least one of the current level of the capability deficits and the current performance level of the patient in the therapy modules, and other information are displayed on the basis of at least one of context and a user's selection.

23. The method as claimed in claim 1, wherein at least one of the patient's capability deficits and therapy modules, at least one of the current level of the capability deficits and the current performance level of the patient in the therapy modules, and other information are displayed on at least one of the basis of the organization unit with which the computer workstation is associated and which is specified by a user.

24. The system as claimed in claim 13, wherein the system is for supporting therapy planning in rehabilitation.

25. The system as claimed in claim 14, wherein at least one of the first and a further database to which the computer workstation is connected contains at least one of a current level of the patient's capability deficits and a current performance level of a patient in the therapy modules, and wherein the module is designed such that it retrieves at least one of the current level of the patient's capability deficits and the current performance level of the patient in the therapy modules, at least one of automatically and on the basis of an input by a user, and is adapted to display it on the computer workstation.

26. The system as claimed in claim 15, wherein at least one of the first and a further database to which the computer workstation is connected contains at least one of a current level of the patient's capability deficits and a current performance level of a patient in the therapy modules, and wherein the module is designed such that it retrieves at least one of the current level of the patient's capability deficits and the current performance level of the patient in the therapy modules, at least one of automatically and on the basis of an input by a user, and is adapted to display it on the computer workstation.

27. The system as claimed in claim 14, wherein at least one of the second and a third database connected to the computer workstation, for a plurality of at least one of capability deficits and therapy modules, contains a fixed link to associated administration information, and wherein the module is adapted to retrieve the administration information at least one of automatically and on the basis of an input by a user and is adapted to display it on the computer workstation in conjunction with at least one of the capability deficits and therapy modules.

28. The system as claimed in claim 15, wherein at least one of the second and a third database connected to the computer workstation, for a plurality of at least one of capability deficits and therapy modules, contains a fixed link to associated administration information, and wherein the module is adapted to retrieve the administration information at least one of automatically and on the basis of an input by a user and is adapted to display it on the computer workstation in conjunction with at least one of the capability deficits and therapy modules.

29. The system as claimed in claim 14, wherein the module is designed such that, on the basis of an input by a user, it is adapted to ascertain an alteration in at least one of the level of the patient's capability deficits and in the performance level of the patient in the therapy modules over time for a prescribable period of time by using the first database, and is adapted to display it on the computer workstation.

30. The system as claimed in claim 15, wherein the module is designed such that, on the basis of an input by a user, it is adapted to ascertain an alteration in at least one of the level of the patient's capability deficits and in the performance level of the patient in the therapy modules over time for a prescribable period of time by using the first database, and is adapted to display it on the computer workstation.

31. A system for supporting therapy planning, comprising:
means for providing at least one of a capability profile, including a plurality of capability deficits, for a patient at the start of a therapy and information about therapy modules prescribed for the patient in the course of therapy from a database;
means for automatically assigning each of the at least one of capability deficits and therapy modules one or more organization units using a second database, wherein the second database includes, for a plurality of at least one of capability deficits and therapy modules, a fixed link to organization units providing therapy; and
means for displaying, on a computer workstation, at least one of capability deficits and therapy modules, the display at least one of including their association and being on the basis of their association on a computer workstation.
